(19) United States
(12) Patent Application Publication
(10) Pub. No.: US 2011/0129449 A1
(43) Pub. Date: Jun. 2, 2011

(54) COMPENSATING FOR ATRIOVENTRICULAR BLOCK USING A NUCLEIC ACID ENCODING A SODIUM CHANNEL OR GAP JUNCTION PROTEIN

(75) Inventors: Ira S. Cohen, Stony Brook, NY (US); Peter R. Brink, Setauket, NY (US); Michael R. Rosen, New York, NY (US); Richard B. Robinson, Cresskill, NJ (US)

(73) Assignee: Columbia University in the City of New York, NYC, NY (US)

(21) Appl. No.: 12/921,395

(22) PCT Filed: Mar. 6, 2009
(86) PCT No.: PCT/US09/36420

§ 371 Date: Feb. 14, 2011

Related U.S. Application Data
(60) Provisional application No. 61/034,879, filed on Mar. 7, 2008.

(51) Int. Cl. A61K 35/12; A61K 48/00; C12N 5/10; A61K 35/76; C07H 21/00; A61P 9/00

(52) U.S. Cl. 424/93.21; 514/44 R; 435/325; 424/93.2; 536/23.5

(57) ABSTRACT

A method of increasing the velocity of AV conduction in a mammal that may be in heart block or at risk of heart block by causing, in an AV node and/or His bundle having less than normal conduction speed, the expression of a sodium channel or gap junction protein, such as the SkM-1 channel, Cx43 or Cx32, so as to increase the velocity of conduction by the AV node.

### CROSS-REFERENCE TO RELATED APPLICATIONS

This application claims priority to United States Provisional Patent Application No. 61/034,879, which was filed on Mar. 7, 2008 and which is incorporated herein by reference in its entirety.

Throughout this application, various publications are referred to. Disclosures of these publications in their entireties are hereby incorporated by reference into this application to more fully describe the state of the art to which this invention pertains. Full bibliographic citations for these references may be found at the end of this application, preceding the claims.

### STATEMENT OF FEDERALLY SPONSORED RESEARCH OR DEVELOPMENT

Work on this invention was sponsored by USPHS and NHLBI under award number HL-28958. Accordingly, the U.S. Government has certain rights in this invention.

### FIELD OF THE INVENTION

The present invention relates to methods and compositions useful in the treatment of atrioventricular (AV) block (also known as heart block) by introduction into and expression in the AV node or His bundle of a nucleic acid that encodes a protein such as the sodium channel SkM-1 or the gap junction protein Cx43 or Cx32, or of cells that express an exogenous such nucleic acid. Recombinant expression of such proteins increases the ability of the AV node to conduct cardiac impulses arising in the sinoatrial node, thereby preventing or alleviating the block.

### BACKGROUND OF THE INVENTION

In a normally functioning human heart, the regular rhythmic pumping of the atria and ventricles is directed and controlled by the heart's electrical system. This system comprises, among other components, the sinus node, which rhythmically generates electrical impulses. Each impulse travels through the atria, resulting in coordinated atrial contraction. Blood is thus pumped from the atria to the ventricles. The atria subsequently relax. Another component of the heart's electrical system, termed the atrioventricular (AV) node, receives the electrical impulse that has travelled through the right atrium and slowly conducts the signal to both ventricles, which subsequently contract. Blood is thus pumped from the right ventricle to the lungs and from the left ventricle to the body. The ventricles subsequently relax. This cycle is repeated 60-100 times per minute in normal adults.

In settings of 2nd or 3rd degree heart block, also known as atrioventricular block or AV block, the atrioventricular node fails to relay or relays only some of the electrical impulses from the normally functioning sinus node to the ventricles. The result can be single dropped beats, multiple dropped beats, or complete loss of conduction from atria to ventricles. As a result, the ventricles pump excessively slowly and do not provide sufficient cardiac output. The outcomes may include syncope (fainting,) ventricular arrest, fibrillation (uncoordinated individual contractions by cardiac cells that fail to pump the blood), and death.

High degree heart block can be caused by coronary atherosclerosis, progressive degeneration of the heart's electrical conduction system and acute myocardial ischemia or infarction. High degree heart block can also be congenital and in some instances related to autoimmune disease in the mother.

Of these causes, acute myocardial infarction (MI) afflicts millions of people each year, inducing significant mortality and, in a large number of survivors, marked reductions in myocyte number and in cardiac pump function. Adult cardiac myocytes divide only rarely, and the usual response to myocyte cell loss is hypertrophy that often progresses to congestive heart failure, a disease with a significant annual mortality. There have been reports of the delivery of mesenchymal stem cells (MSCs, a multipotent cell population of blood lineage) to the hearts of post-MI patients resulting in improved mechanical performance.^{1, 2} In these and other animal studies,³ it is hypothesized that the MSCs integrate into the cardiac syncytium and then differentiate into new heart cells, thus restoring mechanical function.

It is known that fibroblasts can be genetically modified to produce excitable cells capable of electrical coupling by transducing fibroblasts with vectors that encode both the myogenic transcription factor MyoD and Cx43. See E. Kizana et al., Fibroblasts Can Be Genetically Modified to Produce Excitable Cells Capable of Electrical Coupling, Circulation 111: 394 (2005).

High degree heart block currently is treated by installation of an electronic pacemaker (which may be AV sequential if the patient has normal sinus node function). Electronic pacing has drawbacks as it requires the implantation of hardware, consistent monitoring and maintenance including battery and at times lead replacement, the possibility of infection and of interference from other devices. Moreover in children there are issues regarding the growth of the child and mismatches with the lead and pacemaker systems. (See, e.g., M. R. Rosen et al. (2004), Genes, stem cells and biological pacemakers, Cardiovasc. Res. 64: 12-23, M. R. Rosen (2005), Biological pacemaking: In our lifetime? Heart Rhythm 2: 418-428, I. S. Cohen et al. (2005), The why, what, how and when of biological pacemakers. Nat. Clin. Pract. Cardiovasc. Med. 2: 374-375.) There is thus a need for alternative treatments of high degree heart block. The present invention addresses this need by providing a biologically-based method for improving the function of the heart's electrical system.

### SUMMARY OF THE INVENTION

The present invention relates to a method of increasing the velocity of AV node and/or His bundle conduction in a mammal comprising introducing into the cells of the AV node and/or His bundle a nucleic acid molecule that encodes at least one sodium channel or gap junction protein. Such proteins include, but are not limited to, SkM-1, SkM-1-G1306E, Cx43, and Cx32. In the method of the invention, the velocity of AV and/or His bundle conduction after introducing the nucleic acid material is faster than the speed before introducing the nucleic acid material.

The present invention also relates to a method of treating a disorder associated with impaired atrioventricular conduction in a subject's heart comprising introducing into or at a site in close proximity to the cells of the AV node and/or His bundle nucleic acid material that encodes at least one sodium channel or gap junction protein. Such proteins include, but are not limited to, SkM-1, SkM-1-G1306E, Cx43, and Cx32. In the method of the invention, the velocity of AV conduction after introducing the nucleic acid material is faster than the velocity of AV conduction before introducing the nucleic acid material. An embodiment entails determining that the mammal is in heart block or in imminent danger of developing heart block.

The nucleic acid molecule encoding the sodium channel or gap junction protein can be introduced into the cells of the AV node and/or His bundle by contacting the cells of the AV node and/or His bundle with said nucleic acid molecule, or a recombinant expression vector comprising said nucleic acid molecule. Such recombinant expression vectors include, for example, such as an adenovirus or lentivirus or adenoassociated virus vectors. The nucleic acid molecule may be delivered via injection into the AV node or His bundle or into a site near or in close proximity to the AV node.

The present invention also relates to cells of the AV node and/or His bundle wherein an exogenous nucleic acid molecule that encodes a sodium channel, such as SkM-1 or SkM-1-G1306E, or a gap junction protein, such as Cx43 or Cx32, has been introduced into and is expressed in said cells.

The present invention also relates to a method for increasing AV node and/or His bundle conduction in a mammal comprising introduction, delivery, or administration of cells genetically engineered in vitro to express a protein capable of promoting conduction, such as a sodium channel or gap junction protein, to the AV node and/or His bundle in an amount sufficient to increase the velocity of AV and/or His bundle node conduction. Sodium channel proteins include but are not limited to SkM-1 and SkM-1-G1306E. Gap junction proteins include but are not limited to Cx43 and Cx32. The genetically engineered cells to be used in the practice of the invention are preferably mammalian cells. In a preferred embodiment of the invention, the cells are human stem cells, such as human mesenchymal stem cells (hMSCs). The cells may be delivered via an injection into the AV node and/or His bundle or a site near or in close proximity to the AV node and/or His bundle.

The present invention also relates to a kit comprising cells or recombinant expression vectors genetically engineered to express a nucleic acid encoding one or more sodium channel or gap junction proteins. Such proteins include but are not limited to SkM-1, SkM-1-G1306E, Cx43, and Cx32. The kit further comprises a physiologically acceptable carrier for the cells or recombinant expression vector, and directions for administering the cells or recombinant expression vector to a mammal that is in heart block or in imminent danger of developing heart block.

The present invention further provides the use of a nucleic acid molecule that encodes a gap junction protein, or a sodium channel that can be activated at the membrane potential of an AV node in heart block, for example, SkM-1, SkM-1-G1306E, Cx43, and Cx32, to increase the velocity of AV node and/or His bundle conduction, for example by introducing the nucleic acid molecule into the cells of or at a site in close proximity to the AV node and/or His bundle. The use can be applied to a patient suffering from or at risk of suffering from AV block or an associated condition.

The present invention further provides the use of a cell engineered to express a nucleic acid molecule that encodes a gap junction protein, or a sodium channel that can be activated at the membrane potential of an AV node and/or His bundle in heart block, for example, SkM-1, SkM-1-G1306E, Cx43, and Cx32, to increase the velocity of AV node and/or His bundle conduction, for example by introducing the cell into the AV node and/or His bundle or delivering the cells to a site in close proximity to the AV node and/or His bundle. The use can be applied to a patient suffering from or at risk of suffering from AV block or an associated condition.

### DESCRIPTION OF THE DRAWINGS

FIG. 1. Injection of SkM1 adenovirus: ECG leads I and AVF as well as the His bundle electrogram were recorded with A, H and V spikes marked during sinus rhythm, prior to injection.

FIG. 2. Injection of SkM1 adenovirus: ECG leads I and AVF as well as the His bundle electrogram were recorded with A, H and V spikes marked during atrial pacing at CL=500 msec, prior to injection.

FIG. 3. Injection of SkM1 adenovirus: plot of the AH and HV intervals over a range of S2 cycle lengths were recorded before injecting SkM1 adenoviral construct into AV nodal region.

FIG. 4. Injection of SkM1 adenovirus: plot of the AH and HV intervals over a range of S2 cycle lengths were recorded as in FIG. 3 but 7 days after injection. Note no change in the AH interval but the HV interval has shortened.

FIG. 5: FIG. 5 directly compares the HV intervals before injection and 7 days after injection of SkM1. Note that the HV interval has shortened across the full range of cycle lengths.

FIG. 6: Effect of depolarizing the membrane at rest (by increasing external K) on AP upstroke. (A) AP and its first derivative (on an offset, 10× expanded, time scale) from a GFP transfected cell cluster, in 5.4 (left) and 10 mmol/L external K⁺ (right). (B and C) Similar records from Nav1.5-C373Y and SkM1 transfected clusters, respectively. All cells paced at 1 Hz. (D and E) SkM1 but not Nav1.5 expression is protective of AP upstroke in K⁺ depolarized cells. Left: Mean data comparing Vₘₐₓ in GFP and SkM1 (D) or Nav1.5-C373Y (Nav1.5, E) cells. In each case, Na⁺ channel cells were matched to GFP expressing cells from same preparation. Middle: mean data from same cells after raising K⁺ from 5.4 to 10 mmol/L. Right: Mean data on inhibition (as % control) of Vₘₐₓ by elevated K⁺ illustrating the protective effect of SkM1 but not Nav1.5 in comparison to GFP. *P , 0.05 vs. GFP. In (D), n ¼ 10 for both GFP and SkM1 groups; in (E), n ¼ 10 and 13 for GFP and Nav1.5-C373Y, respectively.

### DETAILED DESCRIPTION

Abbreviations used in the specification:
MSC—mesenchymal stem cell;
hMSC—human mesenchymal stem cell;
SKM1—skeletal muscle type 1 sodium channel (also referred to as SCN4a (sodium channel, voltage-gated, type IV, alpha);
Cx—connexin, a gap junction protein.

The present invention provides an in vivo method of increasing the velocity of AV node conduction comprising, or consisting essentially of, or consisting of, introducing into the cells of the AV node and/or His bundle a nucleic acid molecule encoding a sodium channel or gap junction protein, wherein the velocity of AV and/or His bundle conduction after introducing the nucleic acid material is faster than the velocity of AV and/or His bundle conduction before introducing the nucleic acid material. Such nucleic acid molecules include, but are not limited to, those encoding the sodium channel proteins SkM-1 and SkM-1-G1306E and the gap junction proteins Cx43 and Cx32.

In an embodiment of the invention, the nucleic acid material is introduced into the cells of the AV node and/or His bundle through the use of recombinant expression vectors engineered to express a nucleic acid that encodes a sodium channel or gap junction protein of interest. Such vectors include, for example, viral vectors such as adenoviral and adeno-associated viral and lentiviral vectors.

In another embodiment of the invention, cells genetically engineered in vitro to express a nucleic acid that encodes a sodium channel or gap junction protein of interest are introduced into or at a site in close proximity to the AV node and/or His bundle. Such cells include, but are not limited to, mammalian cells, such as human cells, for example mesenchymal stem cells. The sodium channel protein or gap junction protein includes, but is not limited to, SkM-1, SkM-1-G1306E, Cx43, or Cx32. The velocity of AV and/or His bundle conduction after introducing the engineered cells is faster than the velocity of AV conduction and/or His bundle before introducing the cells.

The invention further provides a method of treating a disorder associated with impaired atrioventricular conduction in a subject's heart comprising introducing into the cells of the AV node and/or His bundle nucleic acid material that encodes a sodium channel protein or gap junction protein, such as, but not limited to, SkM-1, SkM-1-G1306E, Cx43, or Cx32, wherein the velocity of AV node and/or His bundle conduction after introducing the nucleic acid material is faster than before introducing the nucleic acid material.

The invention further provides a method of treating a disorder associated with impaired atrioventricular conduction in a subject's heart comprising introducing into or at a site in close proximity to the AV node and/or His bundle cells engineered to express a sodium channel or gap junction protein of interest. Such cells include, but are not limited to, mammalian cells, such as human cells, for example mesenchymal stem cells. The sodium channel protein or gap junction protein includes, but is not limited to, SkM-1, SkM-1-G1306E, Cx43, or Cx32. The velocity of AV and/or His bundle conduction after introducing the engineered cells is faster than the same velocity before introducing the cells.

The present invention provides methods and compositions for providing cells of or in close proximity to the AV node and/or His bundle with a nucleic acid encoding a suitable protein, such as a sodium channel or gap junction protein, for example the sodium ion channels SkM-1 or the variant SkM-1-G1306E or connexins Cx43 and/or Cx32, in order to increase the AV node and/or His bundle conduction velocity, thereby preventing or alleviating arrhythmias caused by AV block. In this way the normal sequence of atrioventricular activation is maintained.

A number of methods can be used to modify the AV node as described in detail below.

In one embodiment of the invention, a nucleic acid molecule encoding an ion channel that can be activated at the low membrane potentials present in the AV node is delivered, in vivo, to AV node and/or His bundle cells. In a specific embodiment, the DNA encodes a sodium channel, including but not limited to the sodium channel encoded by the SkM-1 gene or its variant SkM-1-G1306E.

In another embodiment of the invention, a nucleic acid molecule encoding a gap junction protein is delivered, in vivo, to AV node and/or His bundle cells. In a specific embodiment of the invention, the DNA encodes a connexin, including but not limited to connexin 43 or connexin 32.

A nucleic acid encoding the proteins of interest may be engineered into a variety of host vector systems to direct the expression of said protein in cells of the AV node and/or His bundle. The cDNA sequence and deduced amino acid sequence of the skeletal muscle sodium channel alpha subunit SkM-1 (also known as SCN4A) has been characterized from several species including human, mouse, and rat. Sequences of the SkM-1 proteins are available from public databases. The GenBank ID for human SkM-1 is U24693. This mutant is disclosed at least in N. Mitrović et al. (1995), Different effects on gating of three myotonia-causing mutations in the inactivation gate of the human muscle sodium channel, J. Physiol. 487 (Pt 1):107-14.

The cDNA sequence and deduced amino acid sequence of connexin 43 (Cx43, also known as GJA1) has been characterized from several species including mouse, rat, and human. Sequences of the Cx43 proteins are available from public databases. The GenBank ID for human Cx43 is BCO26329.

The cDNA sequence and deduced amino acid sequence of connexin 32 (Cx32, also known as GJB1) has been characterized from several species including rat, mouse, and human. Sequences of the Cx32 proteins are available from public databases. The GenBank ID for human Cx32 is X04325.

SkM1, Cx43, and Cx32 molecules that fall within the scope of the invention include proteins substantially homologous to the corresponding human form set forth above but derived from another organism, i.e., an ortholog. As used herein, two proteins are substantially homologous when the amino acid sequences are at least about 70-75%, typically at least about 80-85%, and most typically at least about 90-95%, 97%, 98% or 99% or more homologous.

In a specific embodiment of the invention, the SkM1, Cx43, or Cx32 molecule is a SkM1, Cx43, or Cx32 molecule sharing at least 80% homology (as used herein amino acid or nucleic acid “identity” is equivalent to amino acid or nucleic acid “homology”) with the corresponding human form set forth above.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% or more of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The invention also encompasses polypeptides having a lower degree of identity but which have sufficient similarity so as to perform one or more of the same functions performed by SkM1, Cx43, or Cx32. Similarity is determined by considering conserved amino acid substitutions. Such substitutions are those that substitute a given amino acid in a polypeptide by another amino acid of like characteristics. Conservative substitutions are likely to be phenotypically silent. Guidance concerning which amino acid changes are likely to be phenotypically silent are found in Bowie et al., Science 247:1306-1310 (1990).

The comparison of sequences and determination of percent identity and similarity between two polypeptides can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H G., eds., Humana Press, New Jersey, 1994; Sequence Analysis in Molecular Biology, van Heinje, G., Academic Press, 1987; and Sequence Analysis Primer, Gribskov, M. and Devereux, J., eds., M Stockton Press, New York, 1991). A non-limiting example of such a mathematical algorithm is described in Karlin et al. (1993) Proc. Natl. Acad. Sci. USA 90:5873-5877.

In an embodiment, the percent identity between two SkM1, Cx43, or Cx32 amino acid sequences is determined using the Needleman et al. (1970) (J. Mol. Biol. 48:444-453) algorithm. Another non-limiting example of a mathematical algorithm utilized for the comparison of sequences is the algorithm of Myers and Miller, CABIOS (1989).

A substantially homologous SkM1, Cx43, or Cx32, according to the present invention, may also be a polypeptide encoded by a nucleic acid sequence capable of hybridizing to the human SkM1, Cx43, or Cx32, respectively, nucleic acid sequence under stringent conditions, e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3) and encodes a functionally equivalent gene product; or under less stringent conditions, such as moderately stringent conditions, e.g., washing in 0.2×SSC/0.1% SDS at 42° C. (Ausubel et al., 1989 supra), yet which still encodes a functionally equivalent SkM1, Cx43, or Cx32 protein.

In another aspect of the present invention, variant SkM1, Cx43, or Cx32 polypeptides that differ in amino acid sequence by one or more substitutions, deletions, insertions, inversions, fusions, and truncations or a combination of any of these can be used in the treatment methods of the present invention. Variant polypeptides can be fully functional or can lack function in one or more activities.

Nucleotide sequences necessary to recombinantly express the protein of interest, i.e., sodium channel or gap junction proteins, may be isolated using a variety of different methods known to those skilled in the art. For example, a cDNA library constructed using RNA from a tissue known to express the protein of interest can be screened using a labeled probe. Alternatively, a genomic library may be screened to derive nucleic acid molecules encoding the protein of interest. Further, nucleic acid sequences encoding the protein of interest may be derived by performing a polymerase chain reaction (PCR) using two oligonucleotide primers designed on the basis of known nucleotide sequences. The template for the reaction may be cDNA obtained by reverse transcription of mRNA prepared from cell lines or tissue known to express the protein of interest. Nucleic acid sequences that encode mutated forms of the proteins of interest, such as SkM-1-G1306E, can also be prepared by known in vitro mutagenesis methods. The nucleic acid sequence encoding the mutant can be obtained from N. Mitrović et al. (1995) (supra). Methods for preparing nucleic acids can be found in, for example, J. Sambrook et al. (2000), Molecular Cloning: A Laboratory Manual (Third Edition), and Ausubel et al (1996), Current Protocols in Molecular Biology (John Wiley and Sons Inc., USA).

DNA encoding the proteins of interest may be engineered into a variety of host vector systems that also provide for replication and production of the DNA in large scale or contain the necessary elements for directing high level transcription of the nucleotide sequences encoding the proteins of interest. For example, a vector can be introduced in vivo such that it is taken up by a cell and directs the transcription of the nucleic acid molecule encoding the sodium channel or gap junction protein of interest. Such vectors may remain episomal or become integrated into the host genome, as long as it can be transcribed to produce sufficient quantities of the desired protein. Such vectors can be constructed by recombinant DNA technology methods standard in the art.

In instances where a nucleic acid molecule encoding a sodium channel or gap junction protein is utilized, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

Vectors encoding the sodium channels or gap junction protein of interest can be plasmid, viral, or others known in the art used for replication and expression in mammalian cells. Expression of the sequence encoding the proteins of interest can be regulated by any promoter known in the art to act in mammalian, preferably human cells. Such promoters can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42), tetracycline inducible or repressible, ecdysone, mifepristone, or rapamycin promoters, the viral CMV promoter, the human chorionic gonadotropin promoter (Hollenberg et al., 1994, Mol. Cell. Endocrinology 106:111-119), etc. Any type of plasmid, cosmid, YAC or viral vector can be used to prepare the recombinant DNA construct which can be introduced directly into the tissue site.

A selectable mammalian expression vector system can also be utilized to develop stably transformed cells that recombinantly express sodium channels or gap junction proteins. Such cells can be mammalian cells, such as human cells, for example human mesenchymal stem cells (hMSCs). It has been shown that human mesenchymal stem cells can be transfected by electroporation (Hamm, A., et al., (2002), Tissue Eng. 8, 235-245.), among other methods. A number of selection systems can be used, including but not limited to selection for expression of the herpes simplex virus thymidine kinase, hypoxanthine-guanine phosphoribosyltransterase and adenine phosphoribosyl tranferase protein in tk-, hgprt- or aprt-deficient cells, respectively. Also, anti-metabolic resistance can be used as the basis of selection for dihydrofolate transferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin.

In a preferred embodiment of the invention, recombinant viral vectors such as adenoassociated viral or adenoviral vectors may be genetically engineered to express a nucleic acid that encodes a sodium channel or gap junction protein of interest within a selected target cell.

In a particular embodiment of the invention, the nucleic acid molecule encoding the sodium channel or gap junction protein of interest is delivered by a recombinant conditionally replicative adenovirus which is defective for replication but capable of expressing a protein of interest. Within the meaning of the present invention, the expression “conditionally replicative adenovirus” refers to a defective adenovirus which is incapable of autonomous replication in a host cell until the viral defect is complemented in trans.

The present invention encompasses recombinant adenovirus wherein at least one adenovirus gene is deleted. In a preferred embodiment of the invention the adenovirus early region E1, E2 or E4 gene is deleted and replaced with nucleic acid sequences encoding the sodium channel or gap junction protein of interest. Recombinant adenoviruses of the invention also include those viruses having multiple deletions and insertion of one or more sodium channel or gap junction protein-encoding sequences. Since such an adenovirus is conditionally replicative, the virus is initially propagated in cells that complement the deleted region(s) of the adenovirus, i.e., “complementing cell line”. Within the meaning of the present invention “complementing cell line” refers to a cell line that provides the gene products necessary for replication of the defective adenovirus. Such cells include those infected with a helper virus.

In a specific embodiment of the invention, the early region 1 (E1) is deleted and replaced with a nucleic acid sequence encoding a sodium channel or gap junction protein of interest and the virus is propagated in an E1-trans-complementing cell line such as 293 (Graham et al., 1977, J. Gen. Virol. 36:59-72) or in cell lines expressing the pre-mRNA target.

Standard methods for making such deleted adenovirus vectors, such as E1 deleted vectors, may involve in vitro ligation methods or homologous recombination methods. (See Adenoviral Vectors for Gene Therapy, Curiel and Douglas, eds. 2002, Academic Press).

The compositions and methods of the invention can be used to provide sequences encoding a protein of interest to cells of an individual with AV block. The compositions and methods of the invention can be used to increase the velocity of conduction via the AV node and/or His bundle.

Uses And Administration

The compositions and methods of the invention are useful for increasing the velocity of AV conduction. In a preferred embodiment, nucleic acids comprising a sequence encoding a protein of interest are administered to increase the velocity of AV conduction, by way of gene delivery into and expression in a host cell. In this embodiment of the invention, the nucleic acid mediates an effect by promoting sodium channel or gap junction protein production, such as production of SkM1, SkM-1-G1306E, Cx43, or Cx32.

Any of the methods for gene delivery into a host cell available in the art can be used according to the present invention. For general reviews of the methods of gene delivery see Concepts in Gene Therapy, Strauss, M. and Barranger, J. A. eds., 1997, Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; Morgan and Anderson, 1993, Ann. Rev. Biochem. 62:191-217; various authors, 1993, TIBTECH 11(5):155-215. Exemplary methods are described below.

Delivery of the nucleic acid molecule encoding SkM1, SkM-1-G1306E, Cx43, or Cx32 into a host cell may be either direct, in which case the host is directly exposed to the nucleic acid molecule, or indirect, in which case, cells are first transformed with the protein-encoding nucleic acid molecule in vitro, and then transplanted into the host. These two approaches are known, respectively, as in vivo or ex vivo gene delivery.

In a specific embodiment, the nucleic acid or recombinant vector or virus expressing the nucleic acid that encodes the sodium channel or gap junction protein of interest, is directly administered in vivo, where it is taken up by cells of or in close proximity to the AV node and/or His bundle and expressed to produce the protein of interest. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, DuPont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains a nucleic acid encoding a protein of interest, such as a sodium channel or gap junction protein, for example SkM1, SkM-1-G1306E, Cx43, or Cx32-encoding, can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See Kozarsky and Wilson, 1993, Current Opinion in Genetics and Development 3:499-503 for a review of adenovirus-based gene delivery). The vector is designed so that, depending on the level of expression desired, the promoter and/or enhancer element of choice may be inserted into the vector.

Another approach for increasing the velocity of AV conduction is to transfer cells genetically engineered to express a nucleic acid that encodes a sodium channel or gap junction protein to, or to a site in close proximity to, the AV node and/or His bundle. Gene delivery into the cell involves transferring a gene to cells in tissue culture by such methods as electroporation, lipofection, calcium phosphate mediated transfection, or viral infection. Usually, the method of transfer includes the transfer of a selectable marker to the cells. The recombinantly engineered cells are then placed under selection to isolate those cells that have taken up and are expressing the transferred gene. The resulting recombinant cells can be delivered to the AV node and/or His bundle or site in close proximity of a host by various methods known in the art.

In a preferred embodiment, the recombinantly engineered cells to be used for gene delivery are autologous to the host cell. In such an instance, cells may be removed from a subject having AV block or whose electrocardiographic and/or electrophysiologic testing indicate the likelihood of incipient AV block, and transfected with a nucleic acid molecule encoding a protein of interest, for example, a sodium channel or gap junction protein, exemplified by but not limited to SkM1, SkM-1-G1306E, Cx43, or Cx32. Cells may be further selected, using routine methods known to those of skill in the art, for integration of the nucleic acid molecule into the genome thereby providing a stable cell line expressing the protein of interest. Such cells are then transplanted into the AV node and/or His bundle of the subject or a site in close proximity thereto, thereby providing a source of protein capable of promoting AV conduction.

In yet another embodiment of the invention, the transfected cells are MSCs. Human mesenchymal stem cells (Poietics™ hMSCs—Mesenchymal stem cells, Human Bone Marrow) can be purchased from Clonetics/BioWhittaker (Walkersville, Md.) and cultured in MCS growing media.

In an additional embodiment of the invention, cells may be transfected with a nucleic acid encoding one or more of a sodium channel or gap junction protein such as, but not limited to, SkM-1, SkM-1-G1306E, Cx43, and Cx32, so that the protein is functionally expressed in the cells.

The present invention provides a method for increasing the velocity of conduction via the AV node and/or His bundle. The modification of the AV node and/or His bundle in this fashion not only will facilitate propagation of the electrical impulse from atrium to ventricle, but incorporates sufficient delay from atrial to ventricular contraction to maximize ventricular filling and emptying. The goal is to mimic the normal activation and contractile sequence of the heart. Moreover, this approach makes use of an existing physiological system rather than replacing it with an electronic system.

The present invention provides methods and compositions which may be used for treatment of AV block. The term “AV block” as used herein refers to any condition in which conduction via the AV node is slowed, impaired, reduced, or partially or completely lost.

In one embodiment, the present invention provides methods for contacting cells of the AV node and/or His bundle, or sites in close proximity thereto, with a nucleic acid molecule encoding a sodium channel, such as SkM-1 or SkM-1-G1306E, or a gap junction protein, such as connexin 43 or 32, or introducing such a nucleic acid into such cells, by the methods set forth above. Accordingly, the present invention provides a method for treating a subject afflicted with AV block comprising administering such nucleic acids to said subject. The nucleic acids may be administered and/or transplanted to a subject suffering from AV block in any fashion know to those of skill in the art, including by means of viral vectors carrying the nucleic acids.

The present invention further provides methods for contacting cells of or in close proximity to the AV node and/or His bundle with cells engineered to express a nucleic acid molecule encoding a sodium channel, such as SkM-1 or SkM-1-G1306E, or a gap junction protein, such as connexin 43 or 32, by the methods set forth above. Accordingly, the present invention provides a method for treating a subject afflicted with AV block comprising administering such cells to said subject. The cells may be administered and/or transplanted to a subject suffering from AV block in any fashion know to those of skill in the art, including by electrode catheter and/or injection catheter. Cells to be administered include, but are not limited to, mammalian cells, such as human cells, for example human stem cells, for example human mesenchymal stem cells.

Various delivery systems are known and can be used to administer a nucleic acid encoding a protein that can modify AV node and/or His bundle conduction or to administer a cell genetically engineered to modify AV conductance. Such systems may be formulated in any conventional manner using one or more physiologically acceptable carriers optionally comprising excipients and auxiliaries. Proper formulation is dependent upon the route of administration chosen.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington: The Science and Practice of Pharmacy (20th ed. 2000). Such compositions will contain a therapeutically effective amount of the therapeutic compound, preferably in purified form, together with a suitable amount of carrier so as to provide the form for proper administration to the patient. The formulation should suit the mode of administration.

The compositions of the invention can be administered by injection into a target site of a subject, such as the AV node and/or His bundle, preferably via a delivery device, such as a tube, e.g., catheter. In a preferred embodiment, the tube additionally contains a needle, e.g., a syringe, through which the compositions can be introduced into the subject at a desired location.

The compositions may be inserted into a delivery device, e.g., a syringe, in different forms. For example, the compositions of the invention can be suspended in a solution contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art.

The compositions of the invention may be administered systemically (for example intravenously) or locally (for example directly into the AV node and/or His bundle or site in close proximity thereto by means of echocardiogram guidance, or by direct application under visualization during surgery). For such injections, the compositions may be in an injectible liquid suspension preparation or in a biocompatible medium which is injectible in liquid form and becomes semi-solid at the site of damaged tissue. A conventional intra-cardiac syringe or a controllable endoscopic delivery device can be used so long as the needle lumen or bore is of sufficient diameter (e.g. 30 gauge or larger) that shear forces will not damage the cells being delivered.

In a specific embodiment, it may be desirable to administer the compositions of the invention locally to a specific area of the body such as the AV node and/or His bundle or siten in close proximity thereto; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non porous, or gelatinous material, including membranes, such as silastic membranes, or fibers.

The appropriate concentration of the composition of the invention which will be effective in the treatment of

AV block will depend on the nature of the AV block, and can be determined by one of skill in the art using standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses maybe extrapolated from dose response curves derived from in vitro or animal model test systems such as, for example, those set forth in Example 1 of the present specification. Additionally, the administration of the compound could be combined with other known efficacious drugs if the in vitro and in vivo studies indicate a synergistic or additive therapeutic effect when administered in combination.

The progress of the recipient receiving the treatment, including a determination that the velocity of AV conduction has increased after treatment relative to before treatment, can be determined using assays that are designed to test AV node function. Such assays include, but are not limited to electrophysiologic testing and electrical stimulation to determine refractoriness and following frequency at various rates of stimulation.

The present invention also provides a kit comprising:

a) cells genetically engineered to express a nucleic acid that encodes a sodium channel or gap junction protein, wherein said cells, when transplanted into a host AV node and/or His bundle or site in close proximity thereto, provide an increase in conductance, and
b) instructions for introduction of said cells into the

AV node and/or His bundle or site in close proximity thereto.

In a preferred embodiment the cells are mesenchymal stem cells. In another embodiment, the cells are autologous to the host to be treated.

The invention also provides a kit comprising:

a) a nucleic acid molecule encoding a sodium channel or gap junction protein in a pharmaceutically acceptable carrier, and
b) instructions for introduction of the vectors into the AV node and/or His bundle or site in close proximity thereto.

### Example 1

The effect of SkM1 activity in the AV node on cardiac function was measured by introducing SkM1-expressing adenovirus at a site in close proximity to the AV node of a canine heart as identified via a His bundle spike on the intracardiac electrogram. One day before surgery, a 6 lead ECG recording of a conscious dog was conducted to determine PR interval.

On the day of surgery, the dog was anesthetized. A fluoroscopic and electrophysiologic study was performed as follows:
i) A HB recording electrode was passed from right atrium to right ventricle and A-H and H-V intervals were recorded as a standard His bundle recording. A standard electrophysiological study was performed noting usual ECG intervals plus AH-HV intervals, Wenckebach cycle length, higher degree AV block cycle length, and AVN ERP.
ii) Records were made in normal sinus rhythm (NSR) and at two atrial pacing rates, then the maximum following frequency and Wenckebach CL were determined while recording the electrograms.
iii) SkM1 adenovirus was injected into a site near the AV node using 6×10¹⁰ ffu (fluorescent forming units) of virus in a total volume of 1.0 ml.

On days 1, 4, and 6 postoperative a 6 lead ECG in the conscious state was recorded and the PR interval determined.

d) On day 7, steps i) and ii) were repeated.

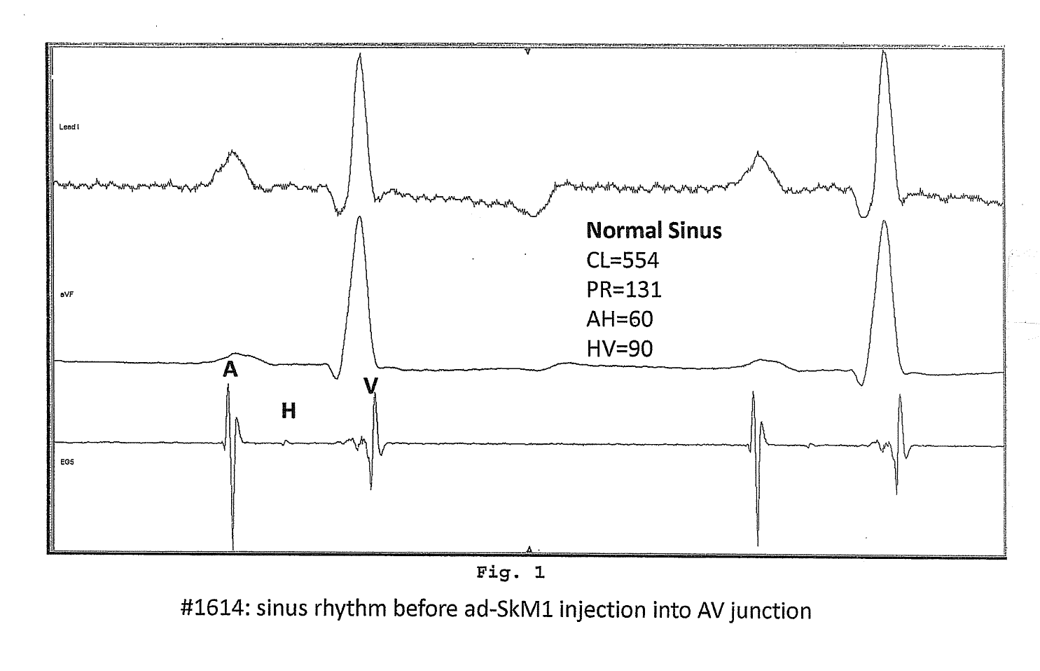

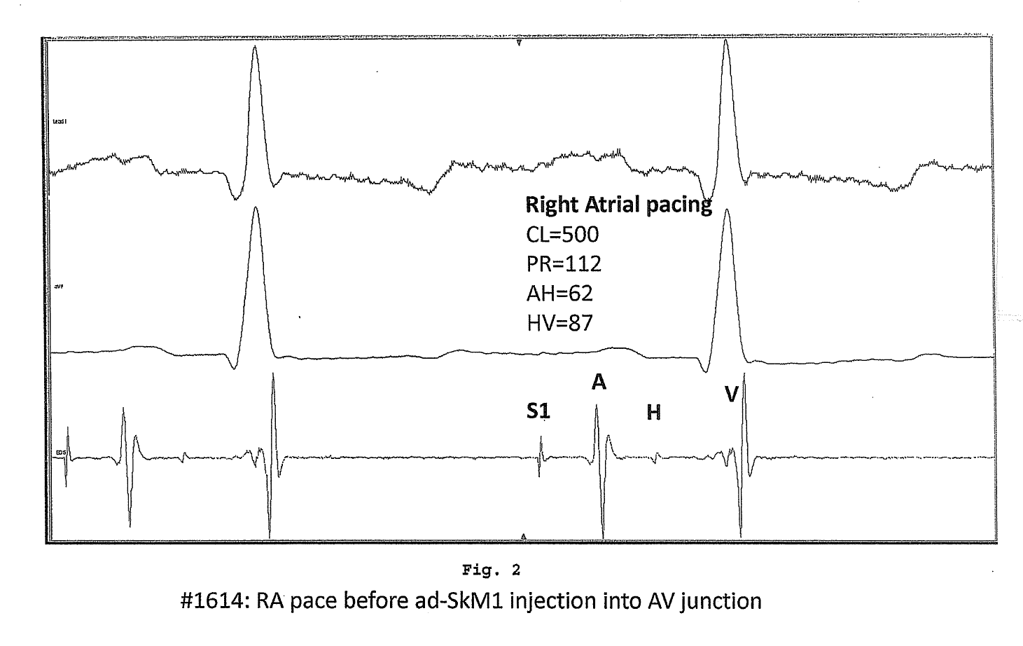

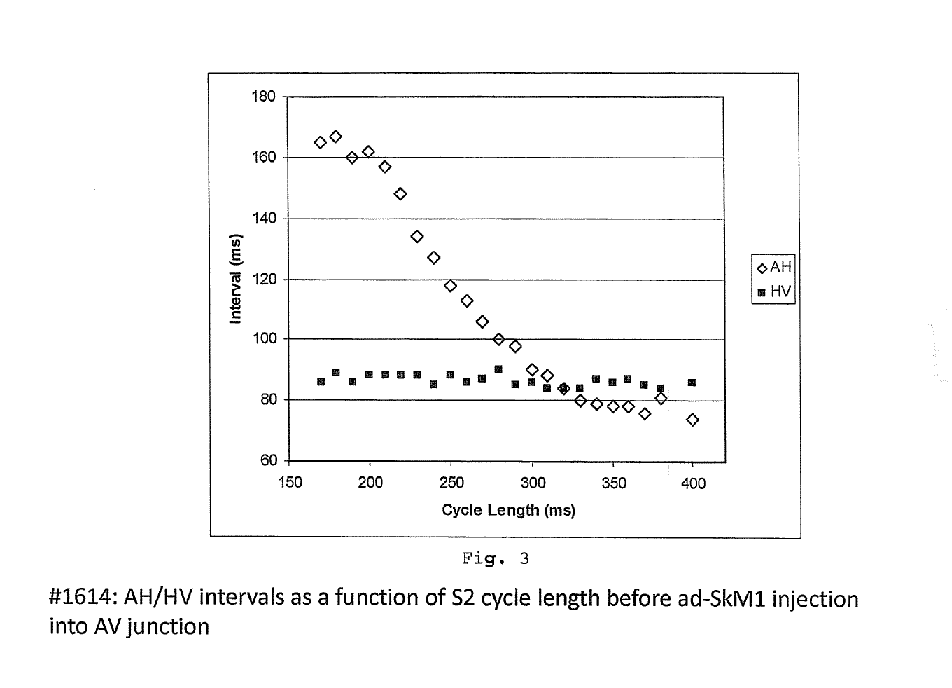

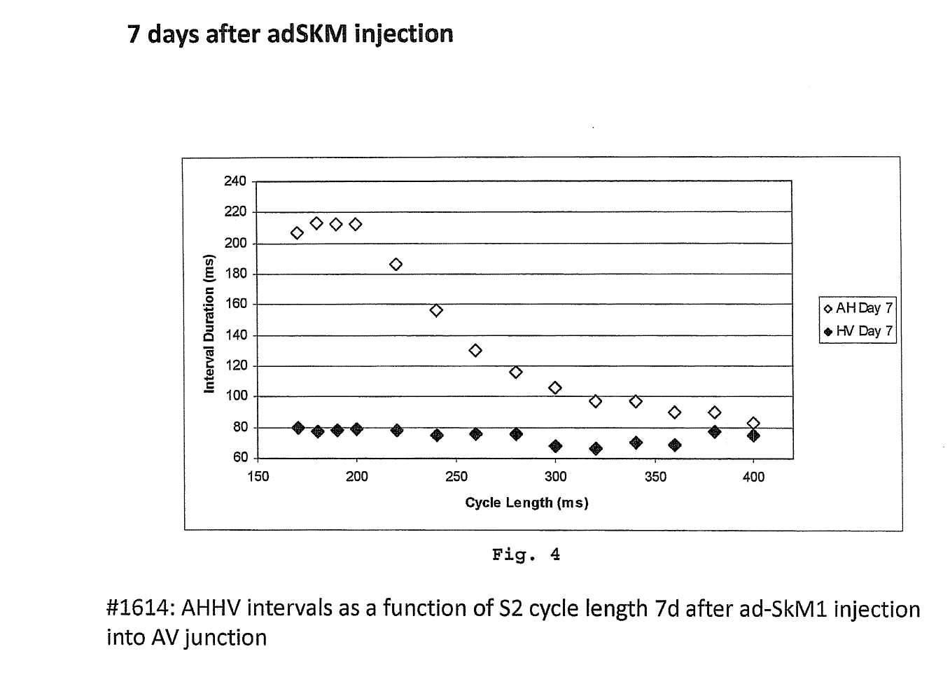

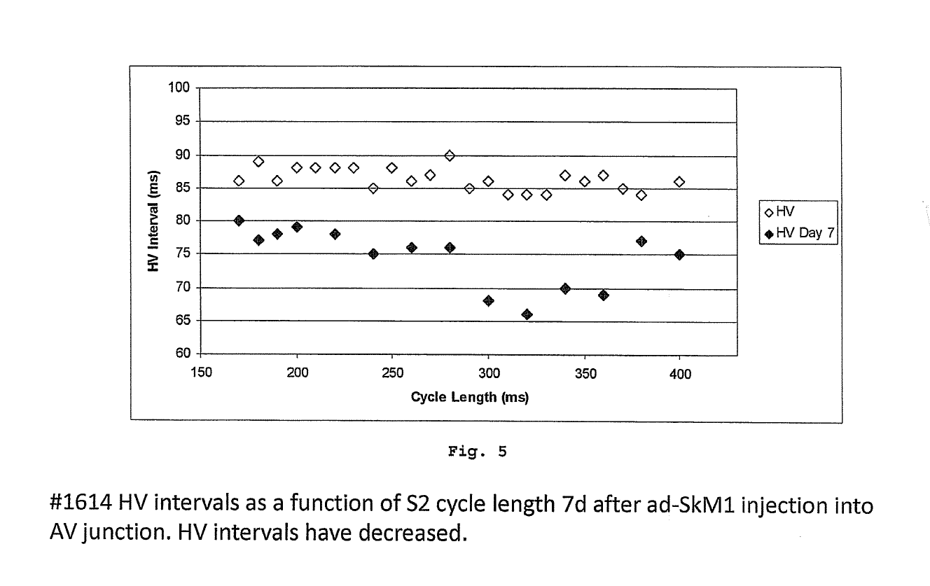

The results are shown in FIGS. [1-5]. FIG. 1 shows ECG leads I and AVF as well as the His bundle electrogram recording with A, H and V spikes marked during sinus rhythm. FIG. 2 shows the same during atrial pacing at CL=500 msec. FIG. 3 plots the AH and HV intervals over a range of S2 cycle lengths before injecting SkM1 adenoviral construct into the AV nodal region. FIG. 4 shows the same intervals as FIG. 3 measured 7 days later. Strikingly, there is no change in the AH interval but the HV interval appears to have shortened. FIG. 5 directly compares the HV intervals before injection and 7 days after injection of SkM1. Notably, the HV interval has shortened across the full range of cycle lengths. This is consistent with speeding of conduction.

### Example 2

### Functional Benefits Of Non-Native Na⁺ Channel Isoform In Newborn Rat Ventricle Cultured Cells

Materials and Methods

Cell Culture

One to two day old rats were sacrificed and the ventricles removed in accordance with Institutional Animal Care and Use Committee Protocols of Columbia and Stony Brook Universities. These studies conform to the Guide for the Care and Use of Laboratory Animals (US National Institutes of Health Publication No. 85-23, revised 1996). Myocytes were isolated using standard enzyme dissociation methods as previously described (Protas & Robinson, Am. J. Physiol. 277:H940-H946 (1999); Yin et al., Am. J. Physiol. Heart Circ. Physiol. 287:H1276-H1285 (2004)). Cells were studied on days 4-6.

For whole cell patch clamp experiments, cells were plated at normal density, then on the experimental day resuspended with 0.1% trypsin and replated as single cells (voltage clamp) or small clusters (AP) for use within 2-8 h. For syncytial studies of propagating APs or CV, cells were plated onto fibronectin-coated coverslips or multi-electrode arrays (MEAs). The MEA array is an 8×8 grid of 30 micron diameter recording electrodes with 200 or 900 micron interelectrode spacing.

Plasmid And Viral Preparation And Gene Expression

To increase the TTX sensitivity of the cloned cardiac Na⁺ channel, a point mutation (C373Y) was introduced into full-length human Nav1.5 cDNA in the pcDNA3.1 plasmid (provided by Dr Robert Kass, Columbia University). This mutation increases TTX sensitivity without altering gating parameters (Satin et al., Science 256:1202-1205 (1992)). The cDNA of the rat skeletal Na⁺ channel SkM1 (provided by Dr Gail Mandel, Oregon Health Sciences) was isolated from its original plasmid and inserted into the shuttle vector pDC516, and an adenovirus prepared from this transgene (Admax system, Microbix, Toronto Canada). The titre of the resulting material was determined using fluorescent focus assay (FFA) with mouse anti-adenovirus antiserum (Advanced ImmunoChemical, Long Beach, Calif., USA) and goat anti-mouse antiserum (Santa Cruz Biotechnology, Santa Cruz, Calif., USA). FFA is an antibody-based titration method which detects only viral particles capable of infecting HEK293 cells. Exogenous genes were expressed in neonatal myocytes by electroporation (Amaxa, Gaithersburg, Md., USA) with ˜30-50% efficiency or adenovirus (>90% efficiency) (Qu et al., Circ. Res. 89:e8-e14 (2001)), using a multiplicity of infection (MOI) of 20 unless otherwise indicated.

Single-Cell Electrophysiological Recordings

APs were recorded using a patch electrode in whole cell mode during superfusion at 35° C. Monolayers were used for studying control cultures and resuspended cells for transfected cultures. Transfected cells were selected by GFP fluorescence (co-transfected with separate GFP vector); adenovirus did not require GFP identification due to high expression efficiency. Cultures were compared to those expressing only GFP virus or plasmid. Extracellular and pipette solutions were as previously employed (Qu et al., Circ. Res. 89:e8-e14 (2001)). External solution contained (mmol/L): NaCl 140, KCl 5.4, CaCl₂ 1, MgCl₂ 1, HEPES 5, glucose 10, adjusted to pH 7.4. Internal solution contained (mmol/L): aspartic acid 130, KOH 146, NaCl 10, CaCl₂ 2, EGTA 5, HEPES 10, MgATP 2, (pH 7.2; 295 mOsm). An Axopatch-200B amplifier, digitizer, and pClamp 8 or 9 software (Molecular Devices, Sunnyvale, Calif.) were used for acquisition and analysis. Stimulation was from a remote extracellular electrode for monolayer studies, and via patch pipette for small clusters. Voltage clamp was conducted on resuspended cells with temperature 19.0 +/−0.5° C. and pipette resistance 1.0-1.5 MΩ for adequate voltage control. Pipette solution contained (mmol/L): CsOH 125, aspartic acid 125, tetraethylammonium chloride 20, HEPES 10, Mg-ATP 5, EGTA 10, and phosphocreatine 3.6 (pH 7.3 with CsOH). After seal formation, stray capacitance was electronically nulled, patch ruptured, and the cell exposed to a low Na⁺ solution (mmol/L): NaCl 50, MgCl₂ 1.2, CaCl₂ 1.8, tetraethylammonium chloride 80, CsCl 5, HEPES 20, glucose 11, 4-aminopyridine 3.0, and MnCl₂ 2.0 (pH 7.3 with CsOH). Currents were filtered at 10 kHz and digitized at sampling interval 0.1 ms for whole cell currents and 0.02 ms for capacitative transients.

Chemicals And Data Analysis

Tetrodotoxin (TTX) was purchased from Calbiochem (Gibbstown, N.J., USA). Statistical analysis was by t-test or ANOVA, as indicated and P<0.05 taken as significant; data are expressed as mean +/− SEM.

Results and Discussion

It was determined whether Na⁺ channel over-expression successfully preserves Vₘₐₓ in elevated external K⁺. In control, non-transfected monolayer cultures, increasing external K⁺ from 5.4 to 10 mmol/L depolarized the resting potential from −75.1+/−1.1 to −57.8+/−1.1 mV (n=8).

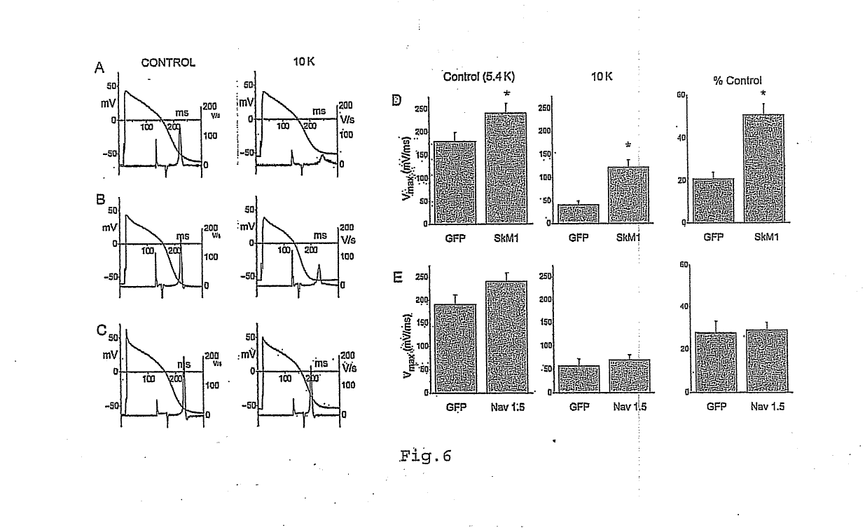

Studies in transfected cells (FIG. 6) demonstrate that SkM1 is more effective than Nav1.5-C373Y in preserving Vₘₐₓ in K⁺ depolarized myocytes. SkM1 increased Vₘₐₓ in both normal and high K⁺ Tyrode compared to GFP; Nav1.5-C373Y provided no benefit in high K⁺ compared to GFP. In addition, no difference was observed in AP amplitude or duration, or on L-type Ca²⁺ current magnitude in SkM1 vs. GFP cells (data not shown). To further confirm that the SkM1 expression was the cause of the protective effect in high K, a separate series of SkM1 and GFP expressing cells (n=7 of each) were first exposed to 100 nmol/L TTX, then to elevated K⁺ in continued TTX. Low TTX significantly reduced Vₘₐₓ in the SkM1 group (from 215+/−13 to 181+/−13 V/s, P<0.05), but not the GFP group (186+/−19 to 171+/−18 V/s, P>0.05). In low TTX and high K⁺ conditions, Vₘₐₓ did not differ between groups (Vₘₐₓ in 10 mmol/L K⁺ 34+/−6 and 37+/−7% of normal K⁺ TTX values, for SkM1 and GFP, respectively; P>0.05) In short, the protective effect of SkM1 was absent in 100 nmol/L TTX.

Thus, in depolarized cells, like those of the AV node, expressing SkM1 increases the rate of action potential depolarization and therefore propagation. The positive inactivation relation of SkM1 is essential to this effect since over-expressing the Nav1.5 cardiac Na channel isoform instead is not effective.

### REFERENCES

1. Strauer, B. E. et al Repair of infarcted myocardium by autologous intracoronary mononuclear bone marrow cell transplantation in humans. Circulation 106, 1913-1918(2002).
2. Perin, E. C., Geng, Y. J. & Willerson, J. T. Adult stem cell therapy in perspective. Circulation 107, 935-938 (2003).
3. Orlic, D. et al. Bone marrow cells regenerate infarcted myocardium. Nature 410, 701-705 (2001).
4. Valiunas, V., Gemel, J., Brink, P. R. & Beyer, E. C. Gap junction channels formed by coexpressed connexin40 and connexin43. Am. J. Physiol. Heart Circ. Physiol. 2001. Oct.; 281. (4.):H1675. -89. 281, H1675-H1689 (2001).
5. Valiunas, V., Bukauskas, F. F, & Weingart, R. Conductances and selective permeability of connexin43 gap junction channels examined in neonatal rat heart cells. Circ. Res. 80, 708-719 (1997).
6. Valiunas, V., Beyer, E. C. & Brink, P. R. Cardiac gap junction channels show quantitative differences in selectivity. Circ. Res. 91, 104-111 (2002).
7. Elfgang, C. et al. Specific permeability and selective formation of gap junction channels in connexin-transfected HeLa cells. J. Cell Biol. 129, 805-817 (1995).
8. Valiunas, V., Weingart, R. & Brink, P. R. Formation of heterotypic gap junction channels by connexins 40 and 43. Circ. Res. 2000. Feb. 4;86.(2.):E42s.- 9.86,E42-E49 (2000).
9. Wang, H. Z. et al. Intercellular communication in cultured human vascular smooth muscle cells. Am. J. Physiol Cell Physiol 281, C75-C88 (2001).
10. Beyer, E. C. Gap junctions. Int. Rev. Cytol. 137C, 1-37 1993).

What is claimed is:

1. A method of increasing the velocity of AV node conduction in a mammal comprising:
introducing into or delivering to a site in close proximity to the cells of the AV node and/or His bundle an effective amount of a nucleic acid molecule that encodes a gap junction protein, or a sodium channel that can be activated at the membrane potential of an AV node in heart block,
wherein the velocity of AV node and/or His bundle conduction after introducing the nucleic acid material is faster than the velocity of AV node and/or His bundle conduction before introducing the nucleic acid material.

2. The method of claim 1, wherein
a) the nucleic acid molecule encodes one or more of the following: SkM-1, SkM-1-G1306E, Cx43, Cx32,
b) the nucleic acid molecule encodes a polypeptide that has at least 80% identity or similarity to human SkM-1, to human SkM-1-G1306E, to human Cx43, or to human Cx32, or
c) the nucleic acid molecule hybridizes under stringent conditions to a nucleic acid molecule that encodes one or more of human SkM-1, human SkM-1-G1306E, human Cx43, and human Cx32.

3. The method of claim 1, further comprising a preliminary step of determining that the mammal is in heart block or at risk of developing heart block.

4. The method of any one of claims 1-3, wherein the nucleic acid material encodes SkM-1.

5. The method of any one of claims 1-3, wherein the nucleic acid material encodes SkM-1-G1306E.

6. The method of any one of claims 1-3, wherein the nucleic acid material encodes Cx43.

7. The method of any one of claims 1-3, wherein the nucleic acid material encodes Cx32.

8. The method of any one of claims 1-3, wherein the nucleic acid material is introduced or delivered by adenoviral vector, or adeno-associated viral construct or lentiviral construct.

9. The method of claim 8, wherein the nucleic acid material encodes SkM-1.

10. The method of claim 8, wherein the nucleic acid material encodes SkM-1-G1306E.

11. The method of claim 8, wherein the nucleic acid material encodes Cx43.

12. The method of claim 8, wherein the nucleic acid material encodes Cx32.

13. A method of treating a disorder associated with an impaired atrioventricular conduction in a subject's heart comprising:
introducing into or at a site nearby the cells of the AV node:
a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
wherein the velocity of AV conduction after introducing the nucleic acid material is faster than the velocity of AV conduction before introducing the nucleic acid material.

14. The method of claim 13, further comprising a preliminary step of determining that the mammal is in heart block or at risk of developing heart block.

15. The method of claim any one of claims 13-14, wherein the nucleic acid material encodes SkM-1.

16. The method of any one of claims 13-14, wherein the nucleic acid material encodes SkM-1-G1306E.

17. The method of any one of claims 13-14, wherein the nucleic acid material encodes Cx43.

18. The method of any one of claims 13-14, wherein the nucleic acid material encodes Cx32.

19. The method of any one of claims 13-14, wherein the nucleic acid material is introduced or delivered by adenoviral vector or adeno-associated viral construct or lentivirus construct.

20. The method of claim 19, wherein the nucleic acid material encodes SkM-1.

21. The method of claim 19, wherein the nucleic acid material encodes SkM-1-G1306E.

22. The method of claim 19, wherein the nucleic acid material encodes Cx43.

23. The method of claim 19, wherein the nucleic acid material encodes Cx32.

24. An AV node cell containing an exogenous nucleic acid molecule that encodes a gap junction protein, or a sodium channel that can be activated at the membrane potential of an AV node in heart block.

25. The AV node cell of claim 24, wherein:
a) the exogenous nucleic acid molecule encodes one or more of the following: SkM-1, SkM-1-G1306E, Cx43, Cx32,
b) the exogenous nucleic acid molecule encodes a polypeptide that has at least 80% identity or similarity to human SkM-1, to human SkM-1-G1306E, to human Cx43, or to human Cx32, or
c) the exogenous nucleic acid molecule hybridizes under stringent conditions to a nucleic acid molecule that encodes one or more of human SkM-1, human SkM-1-G1306E, human Cx43, and human Cx32.

26. A method for increasing AV node conduction velocity in a mammal comprising introducing cells to the AV node or delivering cells to a site in close proximity to the AV node, wherein the cells are engineered to express a nucleic acid molecule that encodes a gap junction protein, or a sodium channel that can be activated at the membrane potential of an AV node in heart block, and wherein the velocity of AV node conduction after introducing or delivering the cells is faster than the velocity of AV node conduction before introducing the cells.

27. The method according to claim 26, wherein the cells are human stem cells.

28. The method according to claim 27, wherein the human stem cells are human mesenchymal stem cells.

29. The method of claim 28, wherein:
a) the nucleic acid molecule encodes one or more of the following: SkM-1, SkM-1-G1306E, Cx43, Cx32,
b) the nucleic acid molecule encodes a polypeptide that has at least 80% identity or similarity to human SkM-1, to human SkM-1-G1306E, to human Cx43, or to human Cx32, or
c) the nucleic acid molecule hybridizes under stringent conditions to a nucleic acid molecule that encodes one or more of human SkM-1, human SkM-1-G1306E, human Cx43, and human Cx32.

30. The method of claim 28, further comprising a preliminary step of determining that the mammal is in heart block or at risk of developing heart block.

31. The method of any one of claims 28 and 30, wherein the nucleic acid material encodes SkM-1.

32. The method of any one of claims 28 and 30, wherein the nucleic acid material encodes SkM-1-G1306E.

33. The method of any one of claims 28 and 30, wherein the nucleic acid material encodes Cx43.

34. The method of any one of claims 28 and 30, wherein the nucleic acid material encodes Cx32.

35. The method of any one of claims 28 and 30, wherein the cells are delivered via an injection into the blood stream, coronary artery, coronary vein, myocardium, pericardial space, or site in close proximity to the AV node.

36. A kit comprising:
a) cells or virus genetically engineered to express a nucleic acid molecule that encodes a gap junction protein, or a sodium channel that can be activated at the membrane potential of an AV node in heart block,
b) a physiologically acceptable carrier for the cells or virus, and
c) directions for administering the cells or virus to a mammal that is in heart block or at risk of developing heart block, and optionally
d) a catheter for administration of the cells or virus.

37. The kit of claim 36, wherein:
a) the nucleic acid molecule encodes one or more of the following: SkM-1, SkM-1-G1306E, Cx43, Cx32,
b) the nucleic acid molecule encodes a polypeptide that has at least 80% identity or similarity to human SkM-1, to human SkM-1-G1306E, to human Cx43, or to human Cx32, or
c) the nucleic acid molecule hybridizes under stringent conditions to a nucleic acid molecule that encodes one or more of human SkM-1, human SkM-1-G1306E, human Cx43, and human Cx32.

38. The kit of claim 37, wherein the nucleic acid material encodes SkM-1.

39. The kit of claim 37, wherein the nucleic acid material encodes SkM-1-G1306E.

40. The kit of claim 37, wherein the nucleic acid material encodes Cx43.

41. The kit of claim 37, wherein the nucleic acid material encodes Cx32.

42. A composition comprising cells or virus genetically engineered to express a nucleic acid molecule that encodes a gap junction protein, or a sodium channel that can be activated at the membrane potential of an AV node in heart block, and a physiologically acceptable carrier for the cells or virus.

43. The composition of claim 42, wherein:
a) the nucleic acid molecule encodes one or more of the following: SkM-1, SkM-1-G1306E, Cx43, Cx32,
b) the nucleic acid molecule encodes a polypeptide that has at least 80% identity or similarity to human SkM-1, to human SkM-1-G1306E, to human Cx43, or to human Cx32, or
c) the nucleic acid molecule hybridizes under stringent conditions to a nucleic acid molecule that encodes one or more of human SkM-1, human SkM-1-G1306E, human Cx43, and human Cx32.

44. The composition of claim 43, wherein the nucleic acid material encodes SkM-1.

45. The composition of claim 43, wherein the nucleic acid material encodes SkM-1-G1306E.

46. The composition of claim 43, wherein the nucleic acid material encodes Cx43.

47. The composition of claim 43, wherein the nucleic acid material encodes Cx32.

48. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32, for the preparation of a medicament.

49. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32, for the preparation of a medicament for use in increasing the velocity of AV node conduction.

50. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32, for increasing the velocity of AV node conduction.

51. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for increasing the velocity of AV node conduction wherein the nucleic acid molecule is introduced into the cells of or at a site nearby the AV node.

52. Use of a cell engineered to express a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for increasing the velocity of AV node conduction wherein the nucleic acid molecule is introduced into the cells of or at a site nearby the AV node.

53. Use of a cell engineered to express a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for increasing the velocity of AV node conduction wherein the cell is introduced into or at a site nearby the AV node.

54. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for the preparation of a medicament for use in treating AV block or an associated condition.

55. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for treating AV block.

56. Use of a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for treating AV block or an associated condition wherein the nucleic acid molecule is introduced into the cells of or at a site nearby the AV node.

57. Use of a cell engineered to express a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for treating AV block or an associated condition wherein the nucleic acid molecule is introduced into the cells of or at a site nearby the AV node.

58. Use of a cell engineered to express a) a nucleic acid molecule that encodes one or more of the following: SkM-1, SkM-1-G1306E, Cx43, Cx32,
b) a nucleic acid molecule that encodes a polypeptide that has at least 80% identity or similarity to human SkM-1, to human SkM-1-G1306E, to human Cx43, or to human Cx32, or
c) a nucleic acid molecule that hybridizes under stringent conditions to a nucleic acid molecule that encodes one or more of human SkM-1, human SkM-1-G1306E, human Cx43, and human Cx32,
for treating AV block or an associated condition wherein the cell is introduced into or at a site nearby the AV node.
